(19) United States
(12) Patent Application Publication
(10) Pub. No.: US 2007/0065351 A1
(43) Pub. Date: Mar. 22, 2007

(54) Transfer apparatus and system, and uses thereof

(76) Inventors: Kenneth Haggerty, Willowdale (CA); Sarah Venhuis, Hamilton (CA)

Correspondence Address: Gowling Lafleur Henderson LLP;Suite 1600, 1 First Canadian Place, 100 King Street West, Toronto, ON, M5X 1G5, CA

(21) Appl. No.: 11/518,877

(22) Filed: Sep. 12, 2006

Related U.S. Application Data
(60) Provisional application No. 60/715,580, filed on Sep. 12, 2005.

(51) Int. Cl. A61K 39/02

(52) U.S. Cl. 422/243

(57) ABSTRACT

A transfer apparatus for facilitating transfer between a higher density fluid and a lower density fluid. The apparatus includes a transfer chamber includes a higher density fluid zone and a lower density fluid zone adjacent each other. A moveable contactor is housed in the transfer chamber. At least a portion of the moveable contactor is moveable between the higher density fluid zone and the lower density fluid zone. A current generator connected to the transfer chamber generates a current in the lower density fluid zone. A transfer system includes a plurality of fluidly connected apparatuses in series.

### FIELD OF THE INVENTION

In one of its aspects, the present invention relates to a transfer apparatus for transfer between a higher density fluid and a lower density fluid. In another of its aspects, the present invention relates to a system for transfer between a higher density fluid and a lower density fluid. In yet another of its aspects, the invention relates to the use of a system for transfer between a higher density fluid and a lower density fluid.

### BACKGROUND OF THE INVENTION

Many processes require a gas/liquid system that includes a large surface area in order to facilitate a reaction or physical-chemical process, referred to generally herein as “transfer”. The transfer of a chemical species between two fluids may be necessary for a number of applications; for example, transfer may be carried out for the purpose of removing a gas from a liquid (stripping), removing a gas from a combined gas flow in order to purify the flow (separation), or transferring the gas to a liquid in order to promote a chemical reaction. In another application, a gas or liquid containing one or more chemical species may be passed over a catalyst in order to promote a chemical reaction.

Often the rate-limiting factor in such fluid-fluid processes is the surface area of the interface between the reacting fluids. While the system of the present invention is suitable for reacting a higher density fluid with a lower density fluid, most typically it will be used to react a liquid with a gas and, consequently, the invention will be described in these terms. Controlling for all other variables, the reaction or transfer rate between a gas and liquid is a function of the ratio of the interface surface area (A) to the liquid flow quantity (volume, V), where greater A/V ratios result in improved reaction or transfer rates.

A further and often limiting factor in such fluid-fluid processes is the time during which the fluids are in contact with each other. The system of the present invention offers the capability to control the contact time together with several other variables such that processes which are uneconomical for short contact times become economical when applied in the present system.

A further and often limiting factor in such fluid-fluid processes is the propensity for flooding or gas hold up at high loading rates. In these circumstances the flow of the fluid through the device is impeded by the flow of the gas (usually counter current). As the flow of the high density liquid and the low density liquid are disconnected in this invention the propensity for hold up is largely eliminated.

A number of devices and arrangements to facilitate the desired contact between a gas and a surface of a liquid are known. Such devices include, for example, packed columns, bubble capped tray columns, spray columns, bubblers and stage contactors. In known devices, high A/V ratios are generally limited by physical constraints. One such constraint is the nature of the media in a packed column: while smaller media produces higher A/V ratios, reducing media size increases the risk of plugging and the associated head loss increase. In an example of another such constraint, bubbled capped tray columns, spray columns, and stage contactors are subject to practical height and hydrodynamic limitations.

Rotating biological contactors (RBCs) are known, and have been used in the treatment of wastewater to provide a support medium for biological growth and aeration for the resulting bacterial populations. Rotating contactors have also been employed for contacting chemicals with the atmosphere, where coincident reactions occur and are facilitated by high rotation speeds.

One gas/liquid process that requires a large transfer surface area is ammonia stripping. Existing ammonia stripping devices encounter efficiency and operational problems when the pH of the ammonia bearing liquid falls below 10. Consequentially, excess base is added in order to maintain stripping efficiencies and, on completion of stripping, it is generally required that the pH be adjusted downward by adding an acid prior to discharging the water.

There is a need in the art for an apparatus that facilitates transfer between fluid flows at low flow rates and at relatively high efficiencies, without the height required for existing fluid contacting devices.

Further, there is a need in the art for an apparatus for ammonia stripping that allows the liquid to be stripped to lower concentrations than existing devices and with a final pH between 7 and 9, with relatively low additional energy consumption, and without the requirement for, and expense of, addition of acids for pH readjustment of the effluent.

Further, there is a need in the art for a system that addresses the problem of efficiently treating several concurrent fluid streams of different concentrations.

### SUMMARY OF THE INVENTION

It is an object of the present invention to obviate or mitigate at least one of the above-mentioned disadvantages of the prior art.

Accordingly, in one of its aspects, the present invention provides a transfer apparatus for facilitating transfer between a higher density fluid and a lower density fluid, the apparatus comprising: a transfer chamber having a higher density fluid zone for receiving the higher density fluid and a lower density fluid zone for receiving the lower density fluid, wherein the higher density fluid zone and the lower density fluid zone are adjacent each other; a contactor mounted in the transfer chamber, at least a portion of which is moveable between the higher density fluid zone and the lower density fluid zone; a current generator connected to the transfer chamber for generating a first current in the lower density fluid zone; a fluid control mechanism for generating a second current in the higher density fluid zone.

In another of its aspects, the present invention provides a transfer system for facilitating transfer between a higher density fluid and a lower density fluid, the system comprising: a plurality of apparatus in fluid communication with one another, each apparatus comprising: a transfer chamber having a higher density fluid zone for receiving the higher density fluid and a lower density fluid zone for receiving the lower density fluid wherein the higher density fluid zone and the lower density fluid zone are adjacent each other; the higher density fluid zone having a higher density fluid inlet and a higher density fluid outlet and the lower density fluid zone having a lower density fluid inlet and a lower density fluid outlet; a contactor mounted in the transfer chamber, at least a portion of which is moveable between the higher density fluid zone and the lower density fluid zone; and a current generator connected to the transfer chamber for generating a current in the lower density fluid zone.

In yet another of its aspects, the present invention provides the use of the present transfer system to strip and/or strip and recover ammonia from a wastewater stream.

In yet another of its aspects, the present invention provides the use of a transfer system of the invention to ozonate a wastewater stream.

In yet another of its aspects, the present invention provides a reactor comprising: a chamber for receiving a fluid to be reacted; and a moveable contactor mounted within the chamber and coated with a catalyst for catalysing the reaction of the fluid.

In a further aspect, the present invention provides a transfer apparatus for facilitating transfer between a higher density fluid located in a high density fluid zone and a lower density fluid located in a low density fluid zone, the apparatus comprising: a contactor, at least a portion of which is moveable between the higher density fluid zone and the lower density fluid zone; and a current generator for generating a first current in the low density fluid zone; and a fluid control mechanism for generating a second current in the higher density fluid zone.

In yet another aspect, the present invention provides a transfer apparatus for facilitating transfer between a higher density fluid and a lower density fluid, the apparatus comprising: a transfer chamber having a higher density fluid zone for receiving the higher density fluid and a lower density fluid zone for receiving the lower density fluid, wherein the higher density fluid zone and the lower density fluid zone are adjacent each other; a contactor rotatably mounted in the transfer chamber, at least a portion of which is moveable between the higher density fluid zone and the lower density fluid zone, the contactor comprising a central core portion operable to allow for the passage of fluid therethrough and including a sheet of inert material wrapped around the outer surface thereof to form a spiral, the inert material being at least partially penetrable by at least one of the lower density fluid and the higher density fluid; a fan connected to the transfer chamber for generating a first current in the lower density fluid zone; and a motor for generating a second current in the higher density fluid zone.

In another aspect, the present invention provides a process for the transfer of a chemical species between a higher density fluid and a lower density fluid comprising the steps of (i) providing a higher density fluid and a lower density fluid; (ii) providing a contactor at least a portion of which is moveable between the higher and lower density fluids and at least a portion of which is partially penetrable by at least one of the higher and lower density fluid; (iii) generating a first current in the lower density fluid; (iv) generating a second current in the higher density fluid, the second current being in the opposite direction to the first current; and (v) moving the contactor between the higher and lower density fluids.

In yet another aspect, the present invention provides a transfer apparatus for facilitating transfer between a higher density fluid and a lower density fluid, the apparatus comprising: a transfer chamber having a higher density fluid zone for receiving the higher density fluid and a lower density fluid zone for receiving the lower density fluid, wherein the higher density fluid zone and the lower density fluid zone are adjacent each other; a series of contactors rotatably mounted in the transfer chamber, at least a portion of the surface of each contactor being moveable between the higher density fluid zone and the lower density fluid zone, and at least partially penetrable by at least one of the lower density fluid and the higher density fluid; a fan connected to the transfer chamber for generating a first current in the lower density fluid zone; a motor for generating a second current in the higher density fluid zone.

### BRIEF DESCRIPTION OF THE DRAWINGS

Embodiments of the present invention will be described with reference to the accompanying drawings, wherein like reference numerals denote like parts, and in which:

FIG. 1 illustrates a schematic sectional view of an embodiment of the present transfer apparatus;

FIG. 2A illustrates a schematic top plan view of an embodiment of the present transfer system showing liquid flow with an optional additional chamber;

FIG. 2B illustrates a schematic top plan view of an embodiment of the present transfer system showing gas flow with an optional additional chamber accommodating gas recirculation;

FIG. 3 illustrates a schematic front view of an embodiment of the present transfer system illustrated in FIGS. 2A and 2B without the optional chamber;

FIG. 4 illustrates a schematic top plan view of an embodiment of the present transfer system showing liquid flow that enables concurrent processing of liquid streams of different concentration;

FIG. 5 illustrates a schematic top plan view of an embodiment of the present transfer system wherein the gas is recirculated through the system;

FIG. 6 illustrates a cross sectional side view of an alternative embodiment of the transfer apparatus of the present invention including a series of fluidly connected chambers each containing a contactor;

FIG. 7 is a cross sectional view of the transfer apparatus of FIG. 6 taken along line C-C;

FIG. 8 illustrates a further embodiment of the contactor of the transfer apparatus of the present invention having a spiral wrapping wound around a central cylinder;

FIG. 9 illustrates the central cylinder of the transfer apparatus of FIG. 8 without the spiral wrapping;

FIG. 10 is a photograph of one embodiment of the central cylinder of the transfer apparatus of FIG. 8; and

FIG. 11 is a photograph of the top view of the transfer apparatus of FIG. 8 enclosed within a housing.

### DETAILED DESCRIPTION OF THE PREFERRED EMBODIMENTS

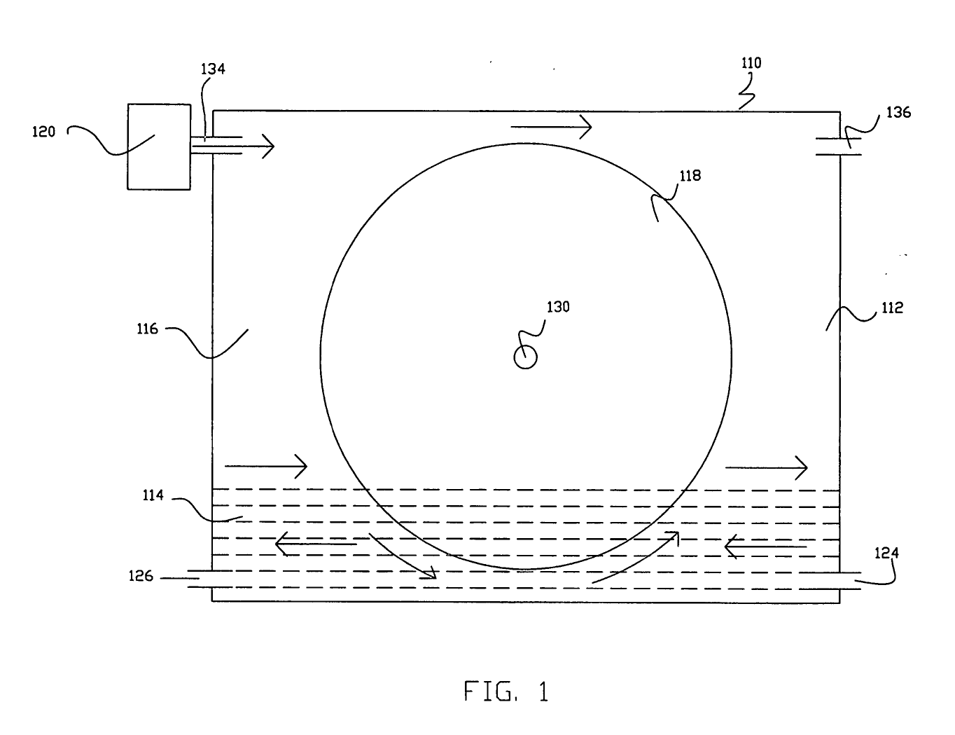

With reference to FIG. 1, there is shown an apparatus 110 of the present invention. Generally, apparatus 110 comprises a transfer chamber 112 containing a higher density fluid zone 114, and a lower density fluid zone 116 positioned adjacent each other. In use, higher density fluid zone 114 will receive a higher density fluid to be treated, while lower density fluid zone 116 will receive a lower density fluid to be treated. In the context of the present application, “treated” will be understood to mean having been passed through the operating transfer apparatus or system such as to allow the desired transfer (e.g., of chemical species) to have occurred. A moveable (e.g., rotating) contactor 118 is housed in transfer chamber 112, and at least a portion of rotating contactor 118 is rotatable between higher density fluid zone 114 and lower density fluid zone 116.

In one embodiment the contactor 118 and the transfer chamber 112 are separate units. In an alternative embodiment the contactor 118 and the transfer chamber 112 comprise a unitary unit and the contactor 118 moves with the transfer chamber 112, preferably by rotation, between the higher and lower density fluid zones.

The depth of the higher density liquid is preferably maintained at a level such that the maximum wetted surface of rotating contactor 118 is exposed to the low density liquid. Rotating contactor 118 is continuously wetted and the drag from its rotation generates mixing in higher density fluid zone 114. A current generator 120 is connected to transfer chamber 112 to generate a first current (shown by arrows) in lower density fluid zone 116. While current generator 120 is shown outside transfer chamber 112, as will be evident to a person skilled in the art, it could be positioned inside transfer chamber 112.

In use, a higher density fluid, typically liquid, and most typically water, is fed into transfer chamber 112 through an inlet 124. For the sake of clarity, the higher density fluid will, in this description, be referred to as liquid, while lower density fluid will be referred to as gas. However, it should be made clear that this is merely a preferred embodiment of how the present apparatus may be used and there may be situations where other combinations of liquid-liquid, gas-liquid and gas-gas may be treated in the present apparatus. In a typical use, the liquid may be untreated drinking water, municipal, residential, agricultural, or industrial wastewater or storm water.

In a preferred embodiment, liquid inlet 124 leads into higher density fluid zone 114, although as will be apparent to a person skilled in the art, liquid inlet 124 may be positioned above higher density fluid zone 114 and the liquid may fall there by gravity. In use, the liquid may be fed into transfer chamber 112 continuously or intermittently. It is preferably fed intermittently when apparatus 110 is constructed on a small scale. Transfer chamber 112 further comprises a higher density fluid outlet or liquid outlet 126 for withdrawing treated liquid. Although while here shown as a separate structure to inlet 124, it will be apparent that a single inlet structure could serve as both inlet and outlet.

A second current is generated in higher density fluid zone 114, which may be intermittent. While various current generators are known to persons skilled in the art, typically, as high density fluid is fed into chamber 112 via inlet 124 or withdrawn from outlet 126, the second current is generated in higher density fluid zone 114. For many loading conditions the efficiency of the apparatus is significantly improved if the second current is in a direction opposite to the first current at the interface of higher density fluid zone 114 and lower density fluid zone 116. The second current in the higher density fluid is typically provided by the external device which introduces the fluid into the device. The fluid preferably transfers within the device by gravity flow.

As will be discussed further below, apparatus 110 may form part of a system comprising a plurality of apparatus 110, preferably connected in series. Where apparatus 110 forms part of such a system, liquid outlet 126 may be a fluid connection to a subsequent apparatus and, preferably, will be a weir. Similarly, where apparatus 110 forms part of a system comprising a plurality of apparatus 110 connected in series, liquid inlet 124 may be a fluid connection to a preceding apparatus, preferably in the form of a weir.

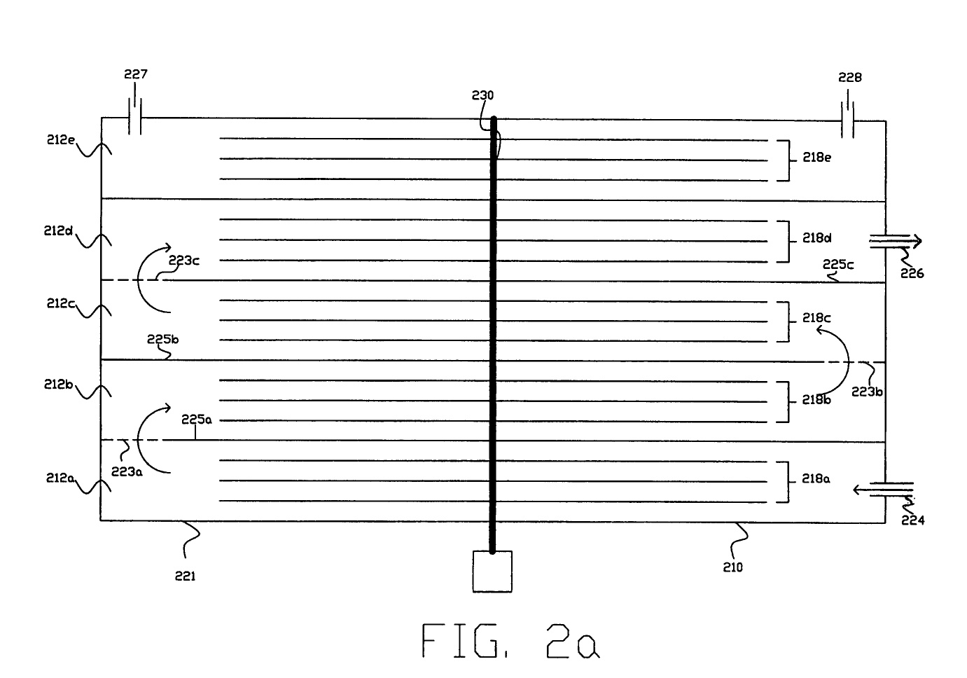

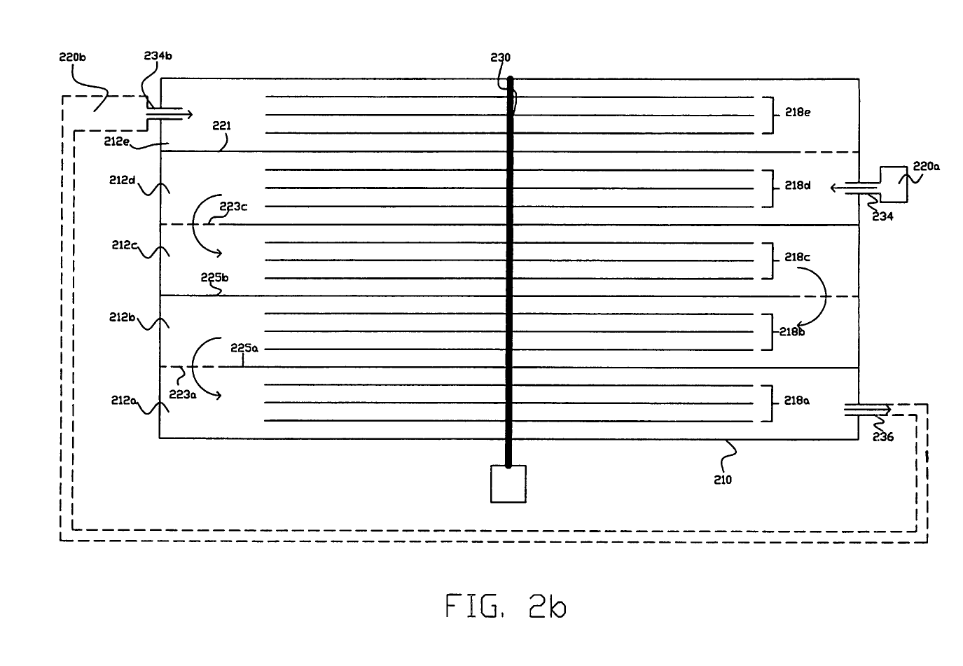

The nature of rotating contactor 118 is not particularly restricted, and the selection thereof is within the purview of a person skilled in the art. Rotating contactor 118 is preferably gas penetrable in that gas can cover and/or pass through a large portion of the contactor surface with low head loss. Further, at least a portion of one or more surfaces of the rotating contactor 118 are partially penetrable by the lower density fluid. The term partially penetrable is used herein to include a situation where at least a portion of the surface is penetrable by the lower density fluid and/or a situation where at least a portion of the surface is periodically penetrable by the lower density fluid, i.e. lower density fluid periodically penetrates a portion of the surface of the contactor. Further, one or more (preferably all) surfaces of rotating contactor 118 are preferably fluid permeable. In one embodiment, rotating contactor 118 comprises a plurality of disks (see FIGS. 2 and 3) or partial disks (not shown) mounted in parallel spaced relation about a common rotatable shaft 130. In this embodiment, gas can pass through the spaces between the disks in the direction of gas flow. Drive means (not shown) rotates shaft 130.

In one embodiment, rotating contactor 118 is a plurality of porous screens, which has a relatively low resistance to gas, and is mounted on a rotating shaft. In yet another embodiment, rotating contactor 118 is a member formed of foamed, extruded, cast, or expanded media, which has a relatively low resistance to gas flow, and provides a large surface area. It will be understood that the contactor 118 may be formed from any inert material and may be provided in any form that includes a surface area that is operable to contact the fluid. It will therefore be understood that the embodiments described above are not meant to be limiting in any way but serve as examples for different types of contactors that may be used.

Gas is fed into transfer chamber 112 via gas inlet 134. As discussed above current generator 120 creates a current within the gas. Current generator 120 is preferably a blower or fan. Transfer chamber 112 further has gas outlet 136. While various inlets and outlets have been shown as discrete structures, it will be apparent to persons skilled in the art that these ports may have dual or multiple functions; an inlet, for example, may be valved so as to operate intermittently as an inlet for one fluid and an outlet for another.

In an exemplary use of the apparatus of the present invention, the gas contains ozone and the liquid is wastewater. Ozone from an ozone source (not shown) is fed into transfer chamber 112 through gas inlet 134. Current generator 120 is suitably a blower for forcing ozone gas from gas inlet 134 under pressure.

Ozone acts as a strong oxidizer to enhance the colour and/or chemical oxygen demand (COD) removal or reduction. Conventional ozone contactors rely on bubbling air containing ozone into a fluid being treated. Where the ozone demand is high and the ozone concentration is low, a significant volume of air must be bubbled into a system in order to meet the ozone demand. Furthermore, the ozone output from many ozone sources is proportionate to the air volume through the generator up to some device dependent maximum.

Rotating contactor 118 facilitates the ozonation of water using a low output ozone source such as an ultraviolet ozone generator (not shown). For a given wastewater, the degree of COD and colour removal when treated with the apparatus of the present invention may be a function of one or more of the quantity of ozone passing over the rotating contactor, the surface area of the rotating contactor, the rotation rate of the rotating contactor, time, liquid characteristics and the temperature. It will be understood by those skilled in the art that whereas ozone represents a reactive gas introduced into the contactor, other gasses may similarly be introduced. Alternatively the contactor may be employed to extract gasses from the liquid by providing a contacting gas with a partial pressure of the gas to be stripped which is lower than the equilibrium partial pressure arising from the gas in the liquid. For example carbon dioxide or weak acids may be stripped from wastewater by applying this principal, for example as seen in Table 2. Under certain conditions this will cause the pH to increase allowing the ammonia to be more readily stripped.

With reference to FIGS. 2A and 2B and FIG. 3, there is schematically illustrated a system 210 of an embodiment of the present invention. In this embodiment, parts identified in the first embodiment are here numbered in the two-hundreds, however, when the same numbers appear as second and third digits, they denote a part corresponding to the part having the same digits in the first embodiment. With reference to FIGS. 2A and 2B, there is shown a system 210 comprising a plurality of chambers 212a, 212b, 212c, and 212d, fluidly connected in series.

A further optional chamber 212e is also shown. As will be apparent to a person skilled in the art, a subsequent chamber or system may be connected either preceding or following the system of the invention in order to carry out a distinct process. In other words, either of lower density fluid or higher density fluid may be selectively passed to a new chamber or system for a new process. In a preferred arrangement, shown in FIGS. 2A and 2B, a chamber 212e receives a flow of gas (but not liquid) from the final (according to gas flow) chamber 212a. In the embodiment shown, optional chamber 212e also feeds gas flow into “first” chamber 212d, and gas is recirculated through system 210 by a device such as a pipe or duct connecting gas outlet 236 with gas inlet 234b. This recirculation may be via any suitable hardware, as will be appreciated by a person skilled in the art, and is shown here schematically as a dashed path. In ammonia stripping and absorption, a typical use, this chamber 212e may contain an absorber/reactant (for example an acidic solution or ion exchange materials). The adsorber/reactant may be withdrawn continuously or intermittently for further processing or storage. Where such adsorber/reactor is withdrawn or consumed it must be replenished in chamber 212e.

The number of transfer chambers 212 connected in series is not particularly restricted and is within the purview of a person skilled in the art in light of the fluid treatment desired. As described above and as shown in FIG. 3, each chamber 212 comprises a higher density fluid zone 214, a lower density fluid zone 216, and a rotating contactor 218a, 218b, 218c, and 218d, each here shown as three disks rotating about common shaft 230. A current generator 220a (or optionally 220b where optional compartment 212e containing an adsorber is included) generates a current flow in the low density fluid zone 216 of each chamber 212. In a preferred embodiment, transfer chambers 212a, 212b, 212c, and 212d are compartments of a larger housing 221. Higher capacity operations may be configured such that chambers 212a, 212b, 212c, and 212d are in linear series, with the contactors operated by either a plurality of drives , or a single device driving all contactor motion.

Liquid shown by the arrows in FIG. 2A is fed into first transfer chamber 212a of system 210. The liquid flows sequentially through chambers 212a, 212b, 212c, and 212d, as shown by the arrows in FIG. 2A, each chamber having a liquid inlet and a liquid outlet.

Preferably, the mechanism employed to allow higher denser liquid transfer between chambers 212a, 212b, 212c, and 212d prevents backmixing between adjacent chambers. Check valves in the fluid interconnection between compartments, or weirs with progressively lower levels are means which successfully achieve this objective. Preferably, transfer chambers 212 are connected by weirs 223a, 223b, and 223c. Weirs 223 are formed by “cutting out” a portion of one of common walls 225a, 225b, and 225c. Generally, the cut-out will be at one end of wall 225 and will extend from the roof of housing 221 to the minimum desired depth of the liquid. As shown in FIG. 2A, transfer chambers 212b and 212c will have a pair of weirs, one operating as a liquid inlet and another operating as a liquid outlet. Preferably, the liquid inlet and liquid outlet weir of a single transfer chamber will be positioned at opposite ends of the chamber. Where higher capacity operations are configured such that chambers 212a, 212b, 212c, and 212d are in linear series, it will be evident to those skilled in the art that hydraulic considerations may result in conditions where backmixing and/or short circuiting is of minimal concern and unimpeded flow between compartments is suitable.

System 210 may be operated continuously, i.e. the liquid is fed continuously into system 210 (a pseudo plug flow condition depending on the number of chambers), or intermittently (in which case a semi-batch kinetic condition exists). Specifically, for a semi-batch operation, a volume of the liquid to be treated is fed into first transfer chamber 212a through liquid inlet 224. Rotating contactor 118 need not be stopped during intermittent liquid feeding for successful operation. The liquid is then transferred from chamber to chamber in series across weirs 223 as a result of the head increase caused by the increase in liquid volume. Similarly, a quantity of treated liquid is recovered through liquid outlet 226 in last transfer chamber 212d. Preferably, liquid outlet 226 is positioned so as to receive a volume of treated liquid substantially corresponding to the volume of liquid fed into the first transfer chamber 212a through liquid inlet 224.

Gas is fed into the system through gas inlet 234, preferably under pressure from current generator 220, or alternatively 220b through gas inlet 234b, where chamber 212e contains an absorber/reactant, and preferably into the last or most downstream transfer chamber 212. (As will be apparent, this chamber will contain the most treated liquid.) The gas can then pass sequentially through weirs 223 in a direction opposite to the flow of liquid, as shown by the arrows in FIG. 2B. Preferably, the gas will pass via the “cut-out” weirs 223a, 223b, and 223c.

As mentioned above in relation to the apparatus of the present invention, the system of the present invention may be used for the treatment of wastewater with ozone gas. The ozone is fed into system 210 through gas inlet 234. Preferably, air and ozone are fed into last chamber 212d. Preferably the air and ozone are introduced by a blower passing air, or oxygen through an ozone generator. Alternatively the contactor may be employed to extract gasses from the liquid by providing a contacting gas with a partial pressure of the gas to be stripped which is lower than the equilibrium partial pressure arising from the gas in the liquid. For example carbon dioxide or weak acids may be stripped from wastewater by applying this principal, for example as seen in Table 2. Under certain circumstances this will cause the pH to increase.

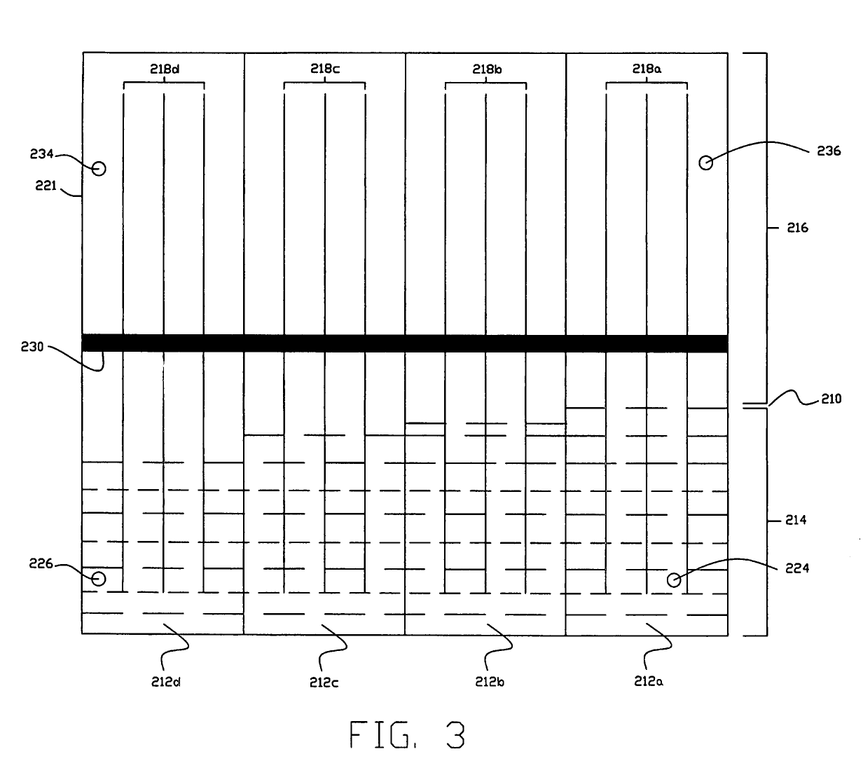

With reference to FIG. 4, there is shown yet another embodiment of the system of the present invention. Here corresponding parts are numbered in the three-hundreds. This Figure shows the flow of liquid. In this embodiment, bypass liquid inlets 338a, and 338b are provided which allow system 310 to be operated partially in parallel. As will be apparent to a person skilled in the art, the position of these bypass inlets is not particularly restricted. Bypass inlets 338 are preferably valved, the system being operable in either the serial or partially parallel manner depending on the treatment objectives. Specifically, for two or more liquid streams of different concentrations, and possibly different flow rates, the liquid with the highest concentration is fed into the first (upstream) compartment via liquid inlet 324. The liquid of the next highest concentration is fed into the downstream compartment via bypass liquid inlet valve 338a, which receives partly treated liquid from the upstream compartment at substantially the same concentration as the less concentrated liquid. By adjusting the locations of the inlets and the number and sizes of the chambers it is possible to tailor the loading of the device to the most efficient configuration for the liquids in question. It will be apparent to those skilled in the art that a reacting species could also be introduced, or withdrawn at the appropriate intermediate points.

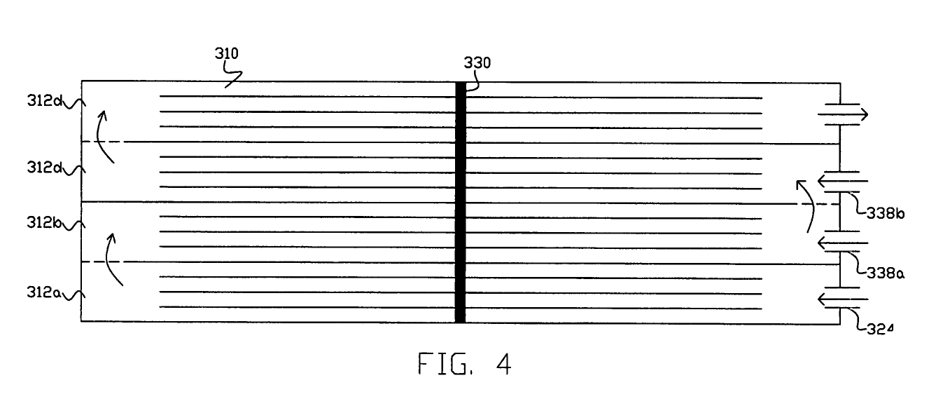

With reference to FIG. 5, there is shown yet another embodiment of the system of the present invention. Here corresponding parts are numbered in the four-hundreds. This Figure shows the flow of gas. In this embodiment, treated gas is recirculated from the last chamber it enters (412a) to the first chamber (412d), via a recirculation tube 440. This aspect of the invention may be combined with other aspects of the invention taught. Similarly, as mentioned above, the gas may be recirculated through an optional adsorbent chamber.

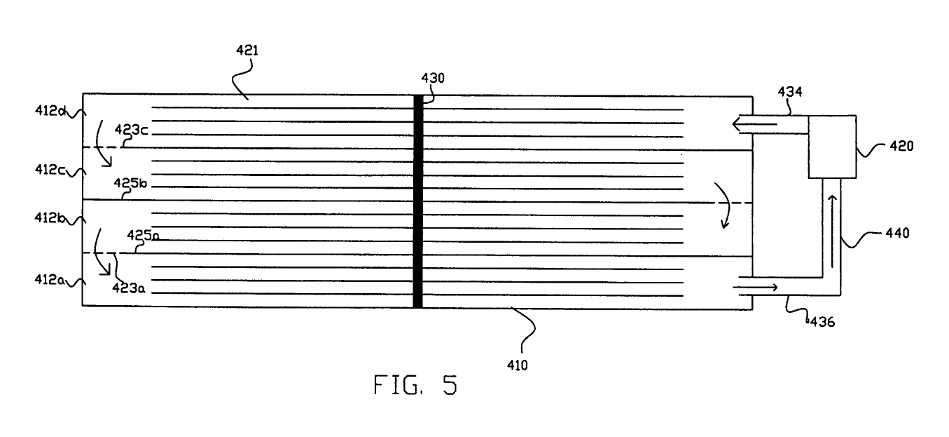

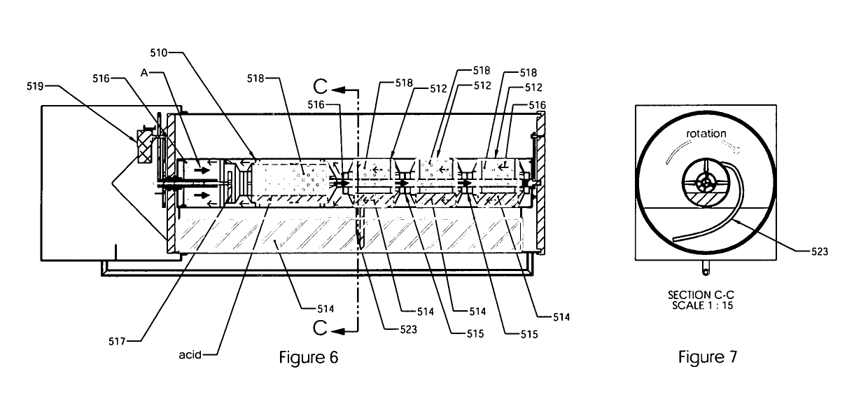

An alternative embodiment of the present invention is shown in FIGS. 6 and 7. The device of the alternative embodiment functions in the same manner as described above while having a different physical embodiment.

Generally in this alternative embodiment the transfer apparatus consists of one or more fluidly connected chambers containing a contactor within each chamber. The chamber or series of chambers is floating or otherwise suspended in a container in which resides the high density fluid and the low density fluid. Turning to FIG. 6 the transfer apparatus is indicated generally at numeral 510 including a series of fluid chambers 512. Within fluid chambers 512 is located a high density fluid zone 514 and a low density fluid zone 516.

The high density fluid 514 is conveyed into each chamber 512 and the chamber 512 is rotated causing the contactor 518 within the chamber to pass through the high density fluid 514, and for the contactor surface to thus be serially covered by the high density fluid. Concurrent with the chamber rotation, the low density fluid 516 flows through the chamber, in the direction of arrows A, allowing the desired interplay between the high density fluid covering the contactor, and the low density fluid 516.

The chambers 512 may be configured such that the high density fluid passes over internal weirs 515 and transfers progressively from one chamber to the next thus producing a cascade effect wherein the composition of the high density fluid will be changed progressively. Alternatively the chambers 512 may be configured such that every revolution of the chamber 512 results in a pumping action wherein a predetermined quantity of the high density fluid moves into and out of the particular chamber 512. Alternatively where hydraulic conditions mitigate against back-mixing no weirs are required.

The contactor 518 in the chamber may be any of the materials described above or alternatively may consist of one of the following: (i) perforated thin inert sheet material wrapped on itself to form a spiral with each wrap separated from the adjacent wraps by a spacer which is preferably created by deformations such as ridges or nodes in the perforated thin inert sheet material, described in further detail below; (ii) disks similar to those described above and extending to the outside walls of the chamber, the disks may also be alternatively perforated near the center of the disks and near the perimeter of the adjacent disks so that the low density fluid passes over the disk surface radially and alternatively moving inward and outward; and (iii) packed media which includes any media having a high surface area to volume ratio, preferably a higher ratio is preferred, however it will be understood that every system will have a limit wherein a higher surface to volume ratio will lead to reduced performance caused by plugging or gas/liquid holdup effects.

As stated above, FIG. 6 illustrates an embodiment of the chambers 512 containing contactors 518 that are operable to rotate. The apparatus 510 is designed to be in a closed tank that preferably contains an ammonia solution. As can be seen in FIGS. 6 and 7, the apparatus 510 includes a fan 517 at the center of one end of the device that serves as a motive force for the low density fluid, i.e. a current generator. The chambers 512 are rotated by a motor 519 located on the same end of the series of chambers 512. As the series of chambers 512 rotate a feeding means or scoop 523 picks up a quanta of high density liquid which then flows progressively through the series of chambers 512 as they rotate. The low density fluid by-passes a portion of the apparatus through a transfer device 521, which may be a supply or return pipe, as illustrated in FIGS. 6 and 7, or similar device that is operable to allow fluid flow, into the area containing the chambers 512, in the direction of arrows A, and returns to the chamber at one end which contains an acid/adsorber-reactant and a contactor which allows the acid to extract the ammonia from the low density fluid which then passes through the device to extract more ammonia. This is functionally the same as pipe 236 in FIG. 2b.

It will be understood that the feeding means or scoop 523 described above is optional and that the feeding means or scoop may be included to assist in the transfer of the high density fluid from the high density fluid zone to the contactor. However, the contactor may not include a feeding means or scoop and the high density fluid may be transferred between the high density fluid zone and the contactor through any appropriate pumping device or mechanism.

In the embodiment described herein, the movement of the contactor 118 is described as being a rotational movement. In the illustrated embodiments, and the description provided, the contactor is operable to rotate a complete 360°. However, it will be understood that the contactor, in the embodiments described herein, need not be operable to rotate a complete 360° or may be operable rotate 360° but in actual operation may only rotate a portion of the full rotational capacity. It will be understood that the rotational movement of the contactor should allow for movement of the contactor to allow at least a portion of the surface to periodically contact at least one of the high and low density fluids. Partial rotation of the contactor within, for example, the high density fluid zone, may allow for sufficient fluid to contact the surface of the contactor and therefore complete rotation may not be required.

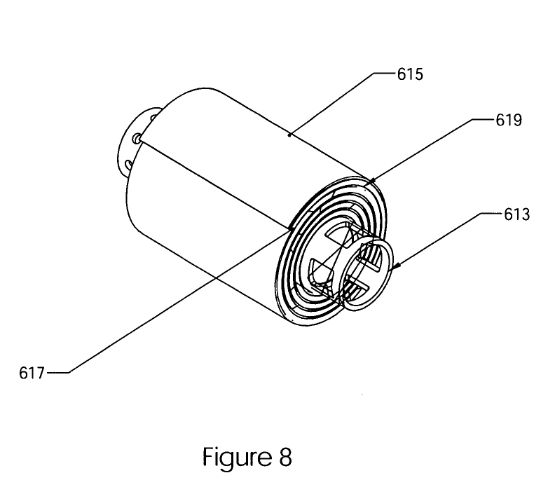

A further alternative embodiment is illustrated in FIGS. 8 through 11. In this embodiment, the fluid transfer apparatus is indicated generally at numeral 610. The apparatus includes a core unit 611 that includes a hollow central cylinder 613 and an inert spiral sheet 615 which together form the contactor 618.

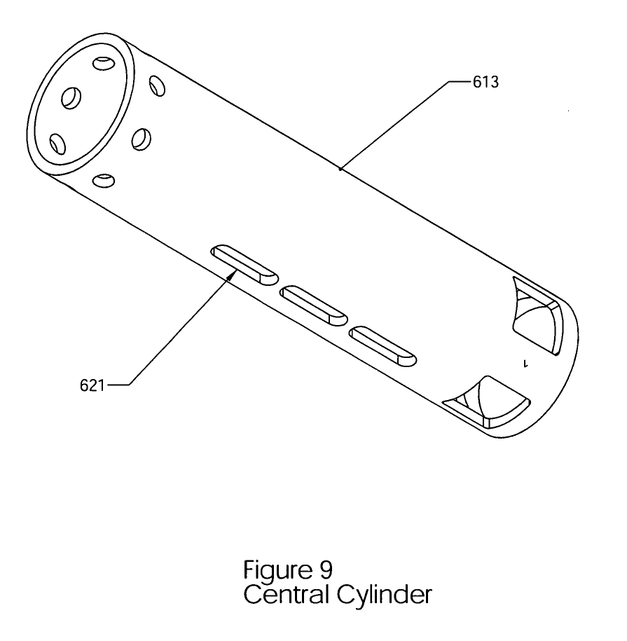

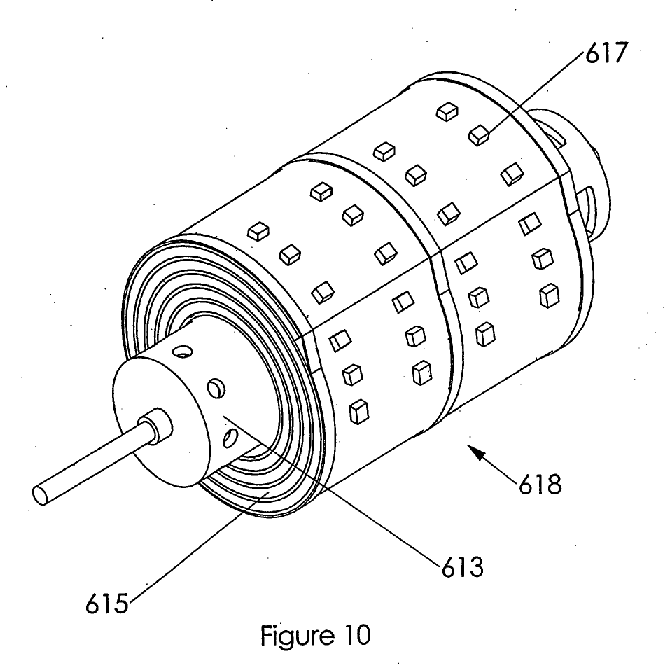

The hollow central cylinder 613 is perforated at each end and along the central axis, as seen in FIG. 9, to facilitate high and low density fluid, e.g. gas and liquid, entry and exit. The inert spiral sheet 615 includes a spacer 617 which maintains a separation between adjacent spirals when the spiral sheet 615 is wound around the central cylinder 613, illustrated in FIGS. 8 and 10.

The spacer 617 may be formed, i.e. integrated, within the surface of the sheet 615, and may be a raised discontinuous surface in the sheet material, preferably created by deformations such as ridges or nodes in the perforated thin inert sheet material. Alternatively the spacer 617 may be one or several separate narrow material strips of a pre-determined thickness that are wound concurrent with the spiral to maintain the desired separation between adjacent sheets 615. The cylindrical spiral so formed is closed on the sides 619, i.e. ends of the spiral section of the roll created by the spiral wrapping, by either an impervious winding or an end cap, to prevent the passage of either the high density fluid or the low density fluid. The result of the above described wrapping is the formation of a sealed helical passage commencing at the end of the thin inert sheet on the outside of the spiral, and ending at the end of the thin inert sheet in the inside of the spiral.

As stated above, the central cylinder 613 is perforated along the portion of the length of the surface where the inert spiral sheet attaches to the cylinder, seen in FIG. 9 at numeral 621. The winding may consist of a single sheet or multiple sheets (with multiple attachment points) which produce nested spirals. In the illustrated embodiment, the spiral windings do not cover the entire length of the central cylinder which projects past the spirals at each end, clearly seen in FIG. 8.

In another embodiment the contactor may be obtained by using semi permeable spiral wrapping, from which the associated “weeping” allows for the A/V ratio to be effectively doubled as both sides of the spiral wrapping are then continuously wetted.

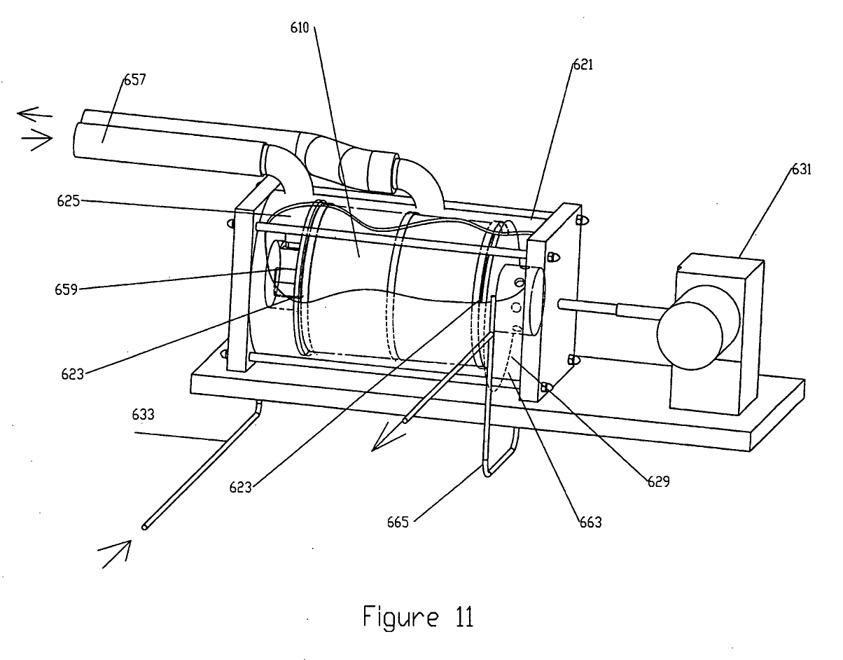

As seen in FIG. 11, when assembled, the contactor 618 is suspended in a tank or gas tight housing 621 containing the high density fluid. The contactor is positioned such that when rotated the open exterior end of the spiral wrapping dips into the high density fluid near the bottom of the contactor 618 and a quantum of the high density fluid enters the contactor 618. The leading edge, i.e. the open exterior end, of the wrapping contacts the liquid and acts as a pump by scooping up a volume (scoop volume) defined by the quantity of liquid pumped into the central chamber since the previous revolution. Alternatively a series of scoops may extend from the leading edge so as to load a prescribed quanta of high density fluid into the spiral with each rotation. Continuous rotation results in a series of quanta of high density fluid being raised towards the interior of the contactor 618 and in the process contacting the surface of the contactor 618. The scoop volume combined with the rate of rotation define the pumping rate. Concurrently the low density fluid is directed into the center of the spiral and passes through the sealed helical chamber until it exits at the perimeter of the spiral. In this way the low density fluid passes over the contactor surface which is progressively in contact with the high density fluid allowing the desired interplay between the high density fluid covering the contactor, and the low density fluid. It is important that an excessive quanta of high density fluid not be added for any one revolution as it can flood the compartment and cause an impediment to gas flow.

When the high density fluid reaches the center of the core 611 it flows to a collector which contains an appropriate device to allow the high density fluid to exit the device without the loss of the low density fluid.

The high density fluid is prevented from flowing into the gas entry section of the device by an internal ring within the central core. By coordinating the liquid pumping rate and the rotation speed of the cylinder 613 the proportion of each winding of the cylinder which is flooded can be controlled, as can the time that the liquid is in the device (HRT). Gas (air) is forced into one end of the central cylinder and flows from the center through the wrappings and out the leading edge of the rotating spiral wrapping. Gas flow rate is one controlled variable. The gas leaves the housing by a duct connected to the housing (generally near the top). This produces a countercurrent gas to liquid flow system which is the most efficient arrangement for mass transfer. The liquid flow has the characteristics of a plug flow device, again this is the most efficient reactor configuration.

The A/V ratio is a function of the number of windings of wrapping material, the surface roughness of the spiral windings and the fraction of the depth of each winding that is flooded which is a function of rotation speed, liquid loading rate and length of the spiral cylinder.

The gas tight housing 621 may be any shape, but in the illustrated embodiment is a hollow cylinder with gas and liquid tight end caps. The portion of the cylinder covered with the spiral wrapping is separated from the ends by a gas seal 623 at each end. The gas seals divide the housing into three sections, the gas entry section 625, the central chamber 627 which contains the core and also serves as the liquid loading section, and the treated liquid section 629.

The drive 631 consists of a device to rotate the core unit, i.e. the central cylinder containing the spiral sheet 617. The drive 631 may take the form of an exterior motor connected to the end of the central cylinder or to a shaft passing through the central cylinder, or the central cylinder may be constructed as an electrical or hydraulic drive unit with an appropriate motive source attached.

The feed system 633 consists of a pre-treatment system, if required, and a pump 635 to deliver the prepared liquid into the central chamber which is normally into the bottom of the chamber.

The gas system consists of a motive device 657 which forces the gas into the gas entry section where it enters the hollow central cylinder through holes/apertures 659 in the cylinder wall, or in the ends of the cylinder.

The treated liquid section 661 consists of the end of the central cylinder opposite to the gas entry cylinder, and an extension of the housing which contains a liquid reservoir 663 and a gas trap 665.

A further alternative embodiment (not shown) involves the incorporation of the spiral pumping arrangement shown in FIGS. 8-11 and described in 0052-0065 into a device with a fluid flow configuration as shown in FIG. 6. In this configuration the sides of the spiral form a barrier to the high density fluid, but are not sealed as described in 52, but contain openings which permit transverse flow of the low density fluid across the spiral in a direction parallel to, rather than perpendicular to, the central axis. This configuration achieves plug flow characteristics for the high density fluid within a single compartment, but not for the low density fluid. Under some circumstances this may be a more economical arrangement.

The kinetics of mass transfer allow for some variations on the general operation described in the above section.

Stripping: The prepared liquid containing the gas to be removed is pumped into the central section. The carrier gas is forced through the spiral where it contacts the liquid and by an application of Henry's Law removes the gas. The gas is ducted out of the Spiral contactor for subsequent treatment.

Adsorption: The operation is identical to the stripping operation except that the gas containing the species to be adsorbed replaces the carrier gas and the adsorbing liquid replaces the liquid containing the gas to be stripped.

Adsorption with a slow reaction: This operation may be conducted in the same manner as the adsorption operation with the difference that a chemical reaction in the adsorbing liquid may determine the operating rate.

Adsorption with a fast reaction: This operation may be conducted in the same manner as the adsorption operation. Alternatively the gas and liquid flow may be co-current as the fast reaction removes any benefits of countercurrent flow.

Stripping and Adsorption with a fast reaction: Two spirals separated by a gas seal may be constructed on the same central core and operated such that the stripped gas is ducted into the housing of the adsorption section where it flows in a co-current direction with the adsorbing reactant liquid. This allows the gas entry section to have a dual function as a spiral contactor with adsorption with fast reaction, as well as the gas entry section.

Catalytic contactor: The surface of the spiral windings may be coated with a catalytic material on one or both sides such that a reaction is catalyzed when the liquid is passed over the surface as a consequence of the pumping produced by the spiral rotation, and/or the gas is catalyzed by a catalyst on the underside of the spiral.

In the case of liquid catalysis, gas flow is optional as determined by the reaction chemistry desired. Alternatively a gas may be passed through the coated spiral and a catalytic reaction produced and liquid flow is optional depending on reaction requirements.

When the underside of the spiral surface is coated with a catalyst and the gas is catalyzed as it passes over this surface, the catalyzed gas may then react with the liquid being pumped thru the spiral contactor by its rotation.

Alternative Operation: If the liquid to be treated is pumped into the center of the spiral and the spiral is rotated in the same direction as the spiral windings the liquid will flow from the inside of the spiral to the outside. Gas flow may then be either co current or counter current as determined by whether the gas is introduced into the center or the perimeter of the device.

One advantage of this device is that it approaches true plug flow in that there is minimal back-mixing of the quanta of high density fluid as it is moved towards the center of the device. In many instances this results in superior process efficiency. By controlling the number of wrappings which compose the spiral and the rotation speed it is possible to control the contact time between the high density fluid and the circulating low density fluid. In the embodiment described above the high density fluid and the low density fluid are fed through the spiral wrapping from opposing ends, i.e. have counter current flow. In an alternative embodiment both the high and low density fluid may be fed into the spiral wrapping at the same position. However, the embodiment described above is preferred.

In each of the above devices the low density fluid may serve as a stripping fluid which may be either wasted or may pass through a separate contacting device for regeneration so that the low density fluid recirculates and a closed system is produced with respect to the low density fluid. Alternatively in instances where the high density fluid acts as an adsorber it may subsequently pass through a regenerator and be recirculated as determined by the optimal process conditions.

One further advantage of the device of the present invention is that it allows for the processing of the dense fluid in time rather than space. This gives the designer/operator significant flexibility in controlling the inputs and the outputs of the device which are not easily obtained from a conventional approach. For example when employed for ammonia stripping the retention time and the pH may be adjusted such that the pH of the water leaving the device is within normal release limits without requiring additional processes to adjust the pH downward after stripping. With conventional stripping processes achieving this is very problematic.

A preferred use of the system of this embodiment of the invention is for stripping and recovering ammonia from a wastewater stream. For stripping ammonia, the stripping rate is a function of the fraction of the ammonia in the gas phase, which is pH and temperature dependant. The equilibrium fraction of ammonia in the gas phase is prescribed by the following relationship:

$$\frac{[ {NH}_{3} ]}{[ {NH}_{3} ] + [ {NH}_{4}^{+} ]} = f = ( {10}^{pKa \text{-} pH} + 1 )$$

where pKa=0.09018+2729.92/T and T=ambient water temperature in Kelvin (K=° C.+273.6). This relationship dictates that at low pH, the ammonia is largely ionized, whereas at high pH it is largely in the unionized state. For example at 20° C. and pH=1, f=4×10−9, whereas at pH=10 and 12, f=0.80 and 0.997, respectively. In the case of the systems tested, it was found that the ammonia stripping rate increased with rotation speed up to about 12-15 revolutions per minute (rpm), after which the increase in stripping rate with increased rpm was much reduced for the species tested (3 gN/L, constant gas flow). However, it will be understood that the rotation speed of the device may be operated at speeds less than 12 or greater than 15 depending on the circumstances under which the device is operated. The above range merely serves as a suggested operational range and is not meant to be limiting in scope.

It will be understood from the above that the rate of, for example, ammonia stripping/adsorption and the extent to which the ammonia can be removed will be a function of at least some of the following variables: (i) The waste water being treated, and the type of adsorber employed; (ii) The pH and alkalinity of the waste water being treated, and the pH of the adsorber if present; (iii) The mechanism/additive employed to control the pH of the wastewater; (iv) The temperature of the waste water being treated; (v) The HRT of the wastewater within the processor; (vi) The wettable surface area of the media within the processor; (vii) The quantity of gas being circulated within the processor; (viii) The aspect ratio (cross-sectional area/Length) and configuration of the processor; and (ix) The rate at which the media is periodically immersed in the waste water.

For an ammonia stripping operation where it is desired to recover the stripped ammonia, the ammonia containing gas is passed over a rotating contactor (preferably on the same drive for small scale units) and immersed in an acid bath preferably at pH less than 4 (i.e. the higher density fluid is an acidic solution), i.e. suitably optional chamber 212e shown in FIGS. 2A and 2B could be used for this purpose. Concurrent with the above-mentioned operations ammonia containing gas is forced over the contactor faces. Those skilled in the art will appreciate that when an acid is employed as the adsorber, it is generally advantageous to ensure that it is neither excessively hydroscopic, nor does it have a high vapour pressure to avoid excessive dilution or evaporation as the case may be..

It will be apparent to a person knowledgeable in reaction kinetics that the flow rates of the different streams and the size of the reaction compartments can be tailored to fit any given set of concentrations and volumes. It is also a feature of this device that it is possible to control the reactor design and operation and the equivalents of base added to the ammonia containing liquids so that the pH within the reactor is adequate for stripping and the pH of the effluent leaving the reactor is between 7 and 9 and does not require the addition of acids for pH adjustment of the effluent prior to further treatment or discharge.

Insulating the apparatus or system of the present invention can eliminate temperature effects from cold surroundings. Recirculation of the stripping gas also mitigates the negative effects of low temperatures.

The example of ammonia stripping and acid absorption can be thought of as contacting with no reaction and contacting with a fast reaction. A number of other processes are possible using the system of the present invention, in addition to those specific processes already described. These processes include:

Absorption with or without a slow reaction: The operation is the reverse of the stripping operation and the gas containing the species to be absorbed is passed over the adsorbing liquid. The operating rate will be determined by absorption rate up to the point where the speed of the chemical reaction is such that it controls the process.

Catalytic reactor: The surface of the media may be coated with a catalytic material such that a reaction is catalysed, or an oxidant is produced, when the liquid is passed over the surface, and/or the catalyst on the disks catalyses a reaction with the gas (for example a semiconductor covered disk may be exposed to air and UV light to produce oxidants). In the case of liquid catalyst, gas flow is optional and determined by the reaction chemistry desired.

Oxidation: A preferred embodiment of this device is to provide a means of contacting an oxidant such as ozone or ultraviolet light and a catalyst with a liquid. Common methods of contacting ozone with a liquid such as bubblers and aspirators are relatively energy intensive, and become very inefficient when dealing with a high ozone demand and a low source concentration of ozone. The RTD can serve as a Rotating Film Oxidizer and can have significant advantages. The effectiveness of the ozonating process is a function of A/V, rotational speed, temperature, and ozone concentration.

Combined Processes: A preferred embodiment of this device is that it offers the possibility of stripping ammonia from a liquid containing ammonia in a series of initial stages of the reactor, ozonating the ammonia stripped liquid and subsequently biologically treating the ozonated liquid within the same device. Further, as will be evident to a person skilled in the art, one or more of the fluids to be treated may be recirculated through one or more treatment systems.

As will be clearly understood from the above description, the present invention provides a device that will allow for ammonia stripping from many waste waters without the usual requirement of the addition of chemicals for pH adjustment. This provides the additional benefit of not requiring such additional chemicals, thereby reducing the chemicals used in the process and the cost.

Further treatment devices may form part of system 210, 310, or 410 before or after the series of transfer chambers 212, 312, or 412. For example, in the treatment of wastewater, the water may be anaerobically and/or aerobically treated in a reactor (not shown) prior to passing through the series of transfer chambers 212, 312 or 412. The wastewater may also be treated upon leaving the last chamber 212d, 312d, or 412d.

Table 1 reports the results obtained treatment of residential wastewater in a device similar illustrated in FIG. 2b, modified to include 7 compartments.

The present invention further provides the use of the transfer apparatus described herein for facilitating transfer of at least one of carbon dioxide, naturally occurring gasses and weak acids from an aqueous wastewater solution into a carrier gas as a means of adjusting the pH. The pH is preferably adjusted to between about 7 and about 10. The pH may be adjusted by the methods described above or by the addition of pH adjusting chemicals.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications and embodiments.

TABLE 1
Example of results from an RTD after ˜18 months treating residential wastewater
consisting of source separated urine and septic tank effluent (STE)

| | Compartment number | | | | | | | |
|---|---|---|---|---|---|---|---|---|
| | 1 | 2 | 3 | 4 | 5 | n.a. | 6 | 7 |
| Liquid Volume, L˜ | 7.5 | 7.1 | 6.8 | 6.5 | 6.1 | n.a. | 6.5 | 6.5 |
| Liquid depth, cm˜ | 14.5 | 14 | 13.5 | 13 | 12.5 | n.a. | 13 | 13 |
| Loading˜batch | 0.5 L | 0 | 0.5 L | 0 | 0 | effluent | 0.5 L | 0.5 L |
| volume added | urine | | STE | | | | acid | acid |
| Batch | 10 | | 9.08 | | | 7.7 | | |
| characteristics pH | | | | | | | | |
| [NH₄ N] | 0.13 | | 0.02 | | | 0.00039 | | |
| Flow in, batch | 1.0 | | 0.33 | | | n.a. | As | As |
| frequency, h | | | | | | | required | required |
| Continuous | 12 | 12 | 12 | 12 | 12 | n.a. | 12 | 12 |
| Rotation rate, rpm˜ | | | | | | | | |
| 40 cm dia. pvc | 6 | 6 | 6 | 6 | 6 | n.a. | 6 | 6 |
| disks per | | | | | | | | |
| compartment | | | | | | | | |
| Air Flow rate, cfm˜ | 80 | 80 | 80 | 80 | 80 | n.a. | 80 | 80 |

TABLE 2
RTD treating liquor from an anaerobic digestor processing residential
organic waste with side stream off-gas to raise pH

| Compartment | Liquor | Acid |
|---|---|---|
| Liquid Volume, L | 2.0 | 1.0 |
| Liquid depth, cm | 10.7 | 5.4 |
| Number of 30 cm diameter disks | 3 | 3 |
| Gas Flow rate, L/min | 390 | 390 |
| Sidestream off-gas flow rate, | 1 | |
| L/min | | |
| Continuous Rotation rate, rpm ˜ | 11 | 11 |
| pH at start | 7.8 | 3.0 |
| PH at end | 8.4 | n.a. |
| Temperature, C. | 19.3 | 19.3 |

Test results

| Elapsed Time, minutes | [N] | pH |
|---|---|---|
| 0 | 0.11 | 7.8 |
| 420 | 0.048 | 8.4 |

What is claimed is:

1. A transfer apparatus for facilitating transfer between a higher density fluid and a lower density fluid, the apparatus comprising:
a transfer chamber having a higher density fluid zone for receiving the higher density fluid and a lower density fluid zone for receiving the lower density fluid, wherein the higher density fluid zone and the lower density fluid zone are adjacent each other;
a contactor mounted in the transfer chamber, at least a portion of which is moveable between the higher density fluid zone and the lower density fluid zone;
a current generator connected to the transfer chamber for generating a first current in the lower density fluid zone; and
a fluid control mechanism for generating a second current in the higher density fluid zone.

2. The transfer apparatus defined in claim 1, wherein the fluid control mechanism comprises an inlet for feeding the higher density fluid into the chamber and an outlet for withdrawing the higher density fluid from the chamber.

3. The transfer apparatus defined in claim 1, wherein when generated, the first current has an opposite direction to the second current at the interface of the lower density fluid zone and the higher density fluid zone.

4. The transfer apparatus defined in claim 2, the first current has an opposite direction to the flow of higher density fluid between the inlet and outlet.

5. The transfer apparatus defined in claim 1, wherein the lower density fluid is a gas.

6. The transfer apparatus defined in claim 5, wherein the higher density fluid is liquid.

7. The transfer apparatus defined in claim 1, wherein the contactor is at least partially penetrable by the lower density fluid.

8. The transfer apparatus defined in claim 7, wherein the contactor comprises a series of spaced discs or partial discs mounted on a common rotatable shaft.

9. The transfer apparatus defined in claim 1, wherein the contactor has fluid permeable surfaces.

10. The transfer apparatus defined in claim 1, wherein the contactor has fluid wetable surfaces.

11. The transfer apparatus defined in claim 10, wherein the contactor is formed of packed media.

12. The transfer apparatus defined in claim 9, wherein the contactor is selected from the group comprising a porous screen mounted on a moveable or rotatable shaft, porous screens mounted on a moveable or rotatable shaft, and a plurality of parallel disc shaped screens mounted on a moveable or rotatable shaft.

13. The transfer apparatus defined in claim 1, wherein the contactor is moveable between the higher density fluid zone and the lower density fluid zone through rotary movement of the transfer chamber in which the contactor is fixed.

14. The transfer apparatus defined in claim 9, wherein the contactor is formed of foamed or expanded media.

15. The transfer apparatus defined in claim 1, wherein the current generator is a blower or fan.

16. The transfer apparatus defined in claim 1, wherein the contactor comprises at least one spiral sheet wound around a central core having a spacer located between overlapping spiral sheet layers.

17. The transfer apparatus defined in claim 16, wherein the spiral sheet is semi-permeable.

18. The transfer apparatus defined in claim 16, wherein the spiral sheet defines an opening at the outer leading edge for contacting at least one of the higher and lower density fluid.

19. The transfer apparatus defined in claim 18, wherein the spiral sheet includes feeding means connected thereto operable to contact at least one of the higher and lower density fluids and feed the fluid to the opening.

20. The transfer apparatus defined in claim 16, wherein the central core comprises at least one aperture for the passage of at least one of the higher and lower density fluids between the central core and the spiral sheet.

21. The transfer apparatus defined in claim 1, wherein the contactor comprises a feeding means connected thereto, operable to transfer high density fluid from the high density fluid zone to at least a portion of the contactor surface.

22. A transfer system for facilitating transfer between a higher density fluid and a lower density fluid, the system comprising:
a plurality of apparatus in fluid communication with one another, each apparatus comprising:
a transfer chamber having a higher density fluid zone for receiving the higher density fluid and a lower density fluid zone for receiving the lower density fluid wherein the higher density fluid zone and the lower density fluid zone are adjacent each other;
the higher density fluid zone having a higher density fluid inlet and a higher density fluid outlet and the lower density fluid zone having a lower density fluid inlet and a lower density fluid outlet;
a contactor mounted in the transfer chamber, at least a portion of which is moveable between the higher density fluid zone and the lower density fluid zone; and
a current generator connected to the transfer chamber for generating a current in the lower density fluid zone.

23. The transfer system defined in claim 22, wherein the higher density fluid passes through the apparatus countercurrent to the lower density fluid.

24. The transfer system defined in claim 22, wherein the lower density fluid is a gas.

25. The transfer system defined in claim 22, wherein the higher density fluid is liquid.

26. The transfer system defined in claim 22, wherein the moveable contactor is at least partially penetrable by the lower density fluid.

27. The transfer system defined in claim 22, wherein the moveable contactor is selected from the group comprising a series of spaced discs mounted on a common rotatable shaft and a series of partial discs mounted on a common rotatable shaft.

28. The transfer system defined in claim 22, wherein the moveable contactor has fluid permeable surfaces.

29. The transfer system defined in claim 22, wherein the moveable contactor has fluid wetable surfaces.

30. The transfer system defined in claim 29, wherein the moveable contactor is formed of packed media.

31. The transfer system defined in claim 29, wherein the contactor comprises a porous screen mounted on a rotatable shaft.

32. The transfer system defined in claim 22, wherein the higher density fluid passes through the chambers continuously.

33. The transfer system defined in claim 22, wherein the higher density fluid passes through the chambers in batches.

34. The transfer system defined in claim 22, wherein at least one outlet of a chamber functions as an inlet of another chamber.

35. The transfer system defined in claim 22, wherein the contactor is affixed to the transfer chamber and rotary motion of the transfer chamber enables the high density fluid to periodically come in contact with a significant portion of the contactor surface.

36. The transfer system defined in claim 22, wherein the contactor comprises at least one spiral sheet wound around a central core having a spacer located between overlapping spiral sheet layers.

37. The transfer system defined in claim 22, wherein the spiral sheet is semi-permeable.

38. The transfer system defined in claim 22, wherein the spiral sheet defines an opening at the outer leading edge for contacting at least one of the higher and lower density fluid.

39. The transfer system defined in claim 38, wherein the spiral sheet includes feeding means connected thereto operable to contact at least one of the higher and lower density fluids and feed the fluid to the opening.

40. The transfer system defined in claim 22, wherein the central core comprises at lease one aperture for the passage of at least one of the higher and lower density fluid between the central core and the spiral sheet.

41. The transfer system defined claim 22, wherein at least one of the transfer chambers has a fluid bypass inlet to enable partial parallel operation of the system.

42. The transfer system defined in claim 22, wherein the lower density fluid is recirculated through the system.

43. The use of the transfer system defined in claim 42 to strip and/or strip and recover ammonia from a wastewater stream.

44. The use of the transfer system defined in claim 22 to ozonate a wastewater stream.

45. The transfer system defined in claim 22, wherein the contactor comprises a feeding means connected thereto, operable to transfer high density fluid from the high density fluid zone to at least a portion of the contactor surface.

46. A catalytic reactor comprising:
a chamber for receiving a fluid to be reacted; and
a moveable contactor mounted within the chamber and coated with a catalyst for catalysing the reaction of the fluid.

47. The catalytic reactor defined in claim 46, wherein the fluid is a liquid that partially fills the chamber and at least a portion of the rotating contactor rotates between the liquid and the surrounding environment.

48. A transfer apparatus for facilitating transfer between a higher density fluid located in a high density fluid zone and a lower density fluid located in a low density fluid zone, the apparatus comprising:
a contactor, at least a portion of which is moveable between the higher density fluid zone and the lower density fluid zone;
a current generator connected to the low density fluid zone for generating a first current in the low density fluid zone; and
a fluid control mechanism in fluid communication with the high density fluid zone for generating a second current in the higher density fluid zone.

49. The use of a transfer apparatus of claim 1 for facilitating transfer of at least one of carbon dioxide, naturally occurring gasses and weak acids from an aqueous wastewater solution into a carrier gas as a means of adjusting the pH.
